(19) United States
(12) Patent Application Publication
(10) Pub. No.: US 2018/0254108 A1
(43) Pub. Date: Sep. 6, 2018

(54) User-Specific Immunogenic Peptide Informed Diet Suggestion Method

(71) Applicant: Jahan Salar Khalili, Everett, WA (US)

(72) Inventor: Jahan Salar Khalili, Everett, WA (US)

(21) Appl. No.: 15/909,940

(22) Filed: Mar. 1, 2018

Related U.S. Application Data
(60) Provisional application No. 62/465,447, filed on Mar. 1, 2017.

(51) Int. Cl. G16H 70/60; G09B 19/00; G16H 70/20; G01N 33/564

(52) U.S. Cl. CPC G16H 70/60; G09B 19/0092; G01N 2800/06; G01N 33/564; G16H 70/20

(57) ABSTRACT

A web-based or mobile application or mobile device is provided that sends and receives data related to genome sequencing, ancestry, and peptide presence in relation to foods and food groups. The application or mobile device will source data either input from the user or inferred by ancestral lineage and determine whether oral tolerance is developed due to antigenic peptides naturally occurring in food. In connection with determining an oral tolerance due to antigenic peptides for an identified food, the application or mobile device may issue an alert or warning.

### CROSS-REFERENCE TO RELATED APPLICATION(S)

The present application claims priority to U.S. Provisional Patent Application No. 62/465,447 filed on Mar. 1, 2017, entitled “User-Specific Immunogenic Peptide Informed Diet Suggestion Method” the entire disclosure of which is incorporated by reference herein.

### BACKGROUND

Recent advances in genotyping, including next-generation sequencing (NGS), polymerase chain reaction, and similar methods have allowed scientists to quickly, effectively, and economically genotype large populations. This has resulted in massive amounts of data in relation to their results. It is now up to scientists to analyze this data and apply it in ways that were either too time consuming, costly, or simply impossible until recently.

This new technology has greatly expanded our understanding of the human genome, including how individuals are predisposed to respond to specific diseases. Humans are known to have about ten thousand Human Leukocyte Antigen (HLA) alleles. The HLA is a gene complex encoding the Major Histocompatibility Complex (MHC) which are cell surface proteins responsible for immune response regulation.

The MHC, specifically MHC class I and MHC class II, binds to antigens which are then present outside of the cell. If the antigen is able to bind to the MHC complex, the antigen will be presented to T-lymphocytes. This is part of a healthy immune response that is needed to help protect against harmful antigens that enter the body. The problem occurs when non-pathogenic antigens are similar enough to the pathogenic antigen to engage a response from the same T lymphocytes. For example, an antigen may be present in a specific food that is similar to a mutated protein of a cancerous cell. Throughout the person's life, they continuously expose themselves to the food antigen to the point which oral tolerance develops, meaning that the immune response no longer responds or has an attenuated response due to repeated exposure to the antigen in tolerizing peripheral tissues such as the digestive tract. This only becomes a problem when the antigen is not unique to the non-pathogenic source, i.e. food, and is also generated by a pathogenic element, such as a tumor or virus infected cell. When this occurs, the immune system will not generate a response against the cancer cells that is as effective at preventing the progression or mediating regression of the cancer, allowing the disease to progress in the absence of the most potent immune response possible.

With this in mind, and with newly found tools such as ancestry data, next-generation sequencing, among others, it is possible to predict the HLA alleles of an individual with varying degrees of certainty. If it is found that the user has a specific allele encoding a conformation of the MHC class complexes that will selectively bind to an antigen present in a specific food item, it is possible to identify other pathogenic antigen sources and alert the person. A suitable solution is desired.

Based on the foregoing, there is a need in the art for a user-friendly mobile application that is able to correspond data from multiple sources in order to develop a diet, or suggest food options and alternatives that avoid oral tolerance resulting from user (e.g. the consumer) specific high-risk foods.

### BRIEF DESCRIPTION OF THE DRAWINGS

For a more complete understanding of the present foregoing, the objects and advantages thereof, reference is now made to the ensuing descriptions taken in connection with the accompanying drawings briefly described as follows.

FIG. 1 is a flowchart of the authentication method, according to an embodiment;

FIG. 2 is a flowchart of the personalization method, according to an embodiment;

FIG. 3 is a flowchart of the genome settings method, according to an embodiment;

FIG. 4 is a flowchart of the personalization method, according to an embodiment;

FIG. 5 is a flowchart of the personalization method, according to an embodiment;

FIG. 6 is a flowchart of the personalization method, according to an embodiment;

FIG. 7 is a flowchart of the purchasing orders method, according to an embodiment;

FIG. 8 is a flowchart of the universal reporting method, according to an embodiment;

FIG. 9 is a flowchart of the ancestry reporting method, according to an embodiment;

FIG. 10 is a flowchart of the prevention reporting method, according to an embodiment;

FIG. 11 is a flowchart of the oncology reporting method, according to an embodiment;

FIG. 12 is a flowchart of the react reporting method, according to an embodiment;

FIG. 13 is a flowchart of the genetically defined ingredients method, according to an embodiment;

FIG. 14 is a flowchart of the ingredient explorer method, according to an embodiment; and

FIG. 15 is a representation of a mobile device implementing the methods herein, according to an embodiment.

### DETAILED DESCRIPTION OF EMBODIMENTS

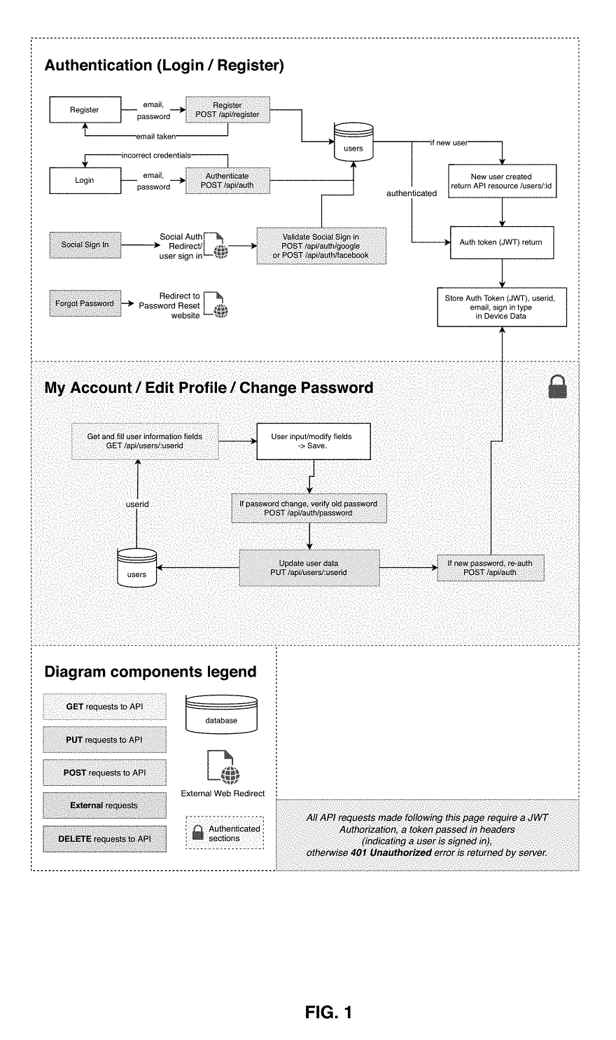

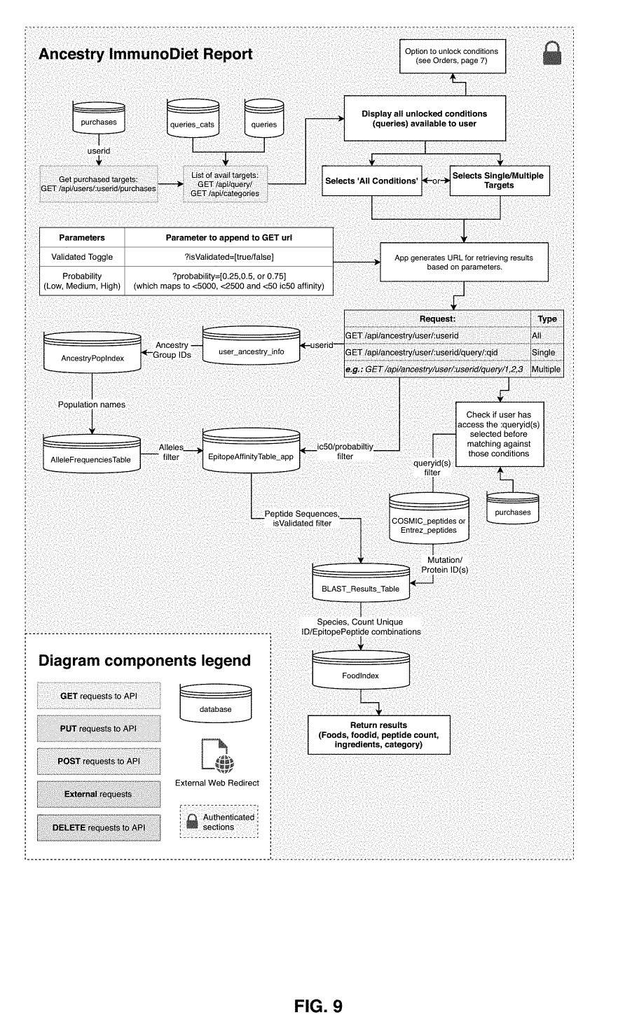

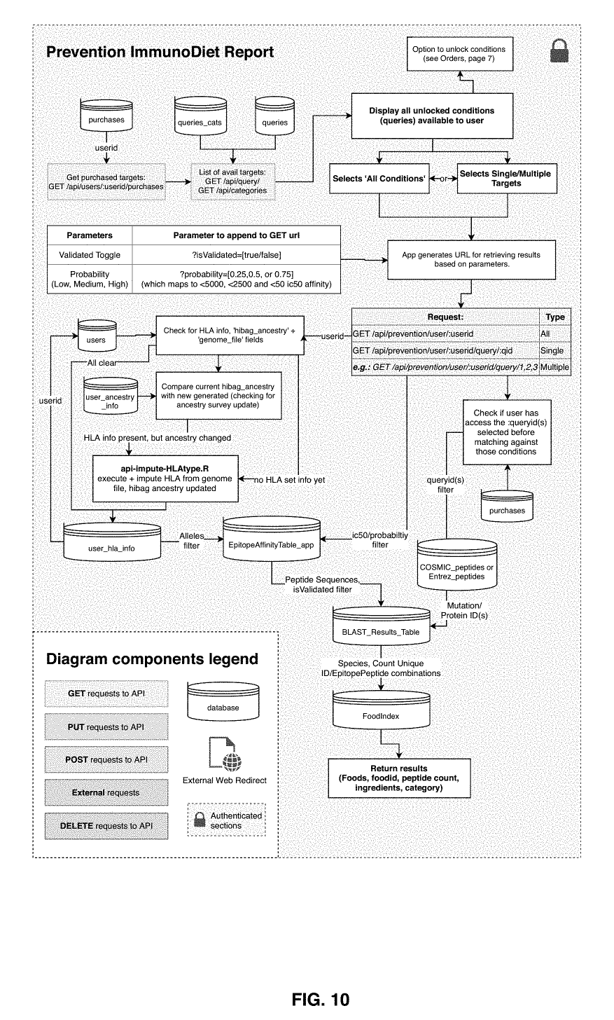

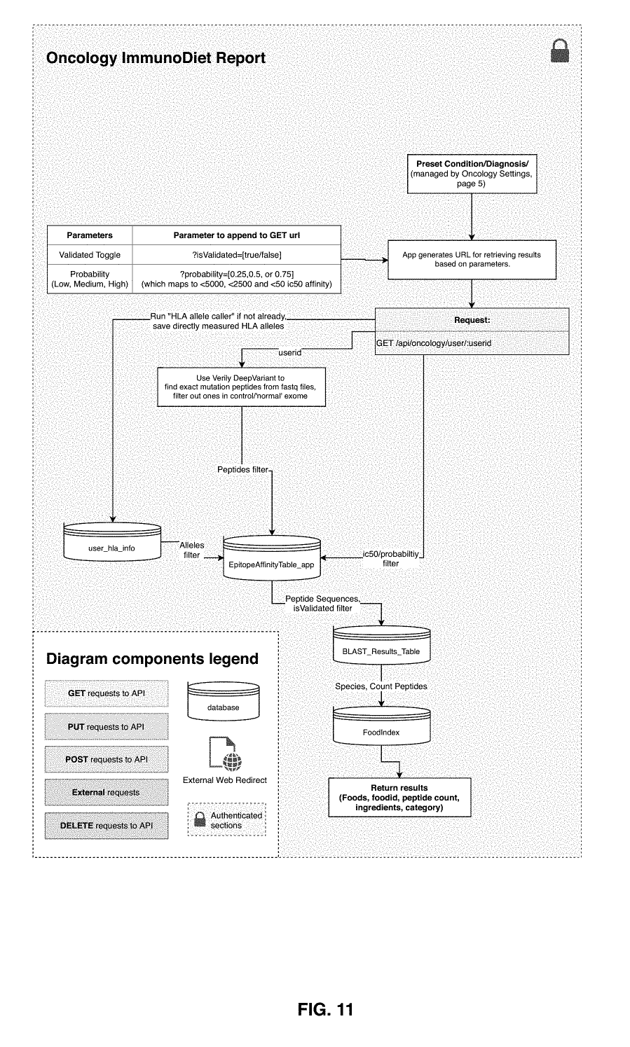

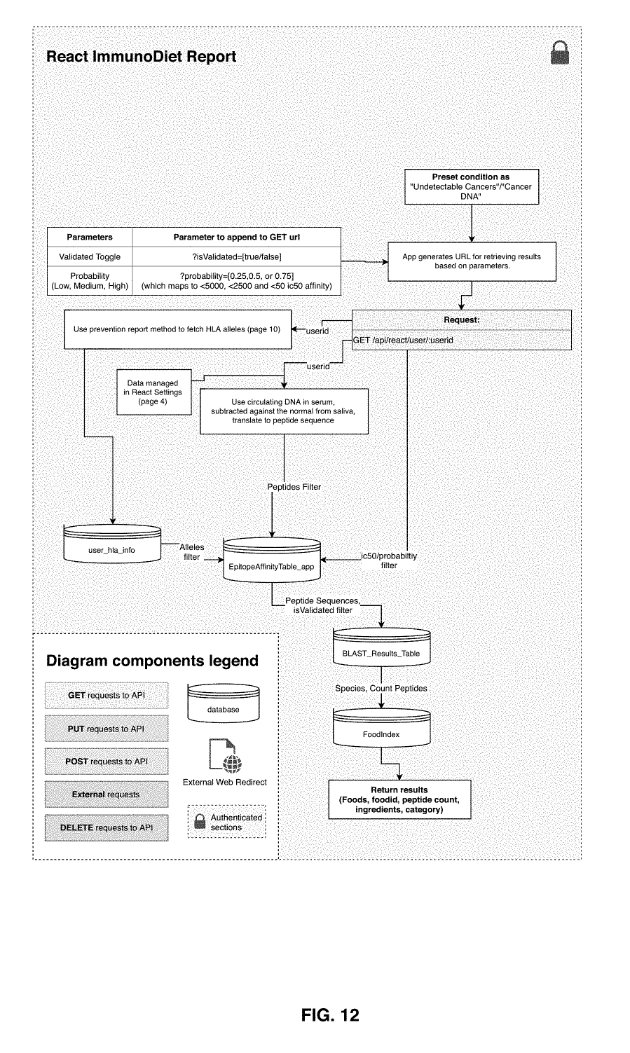

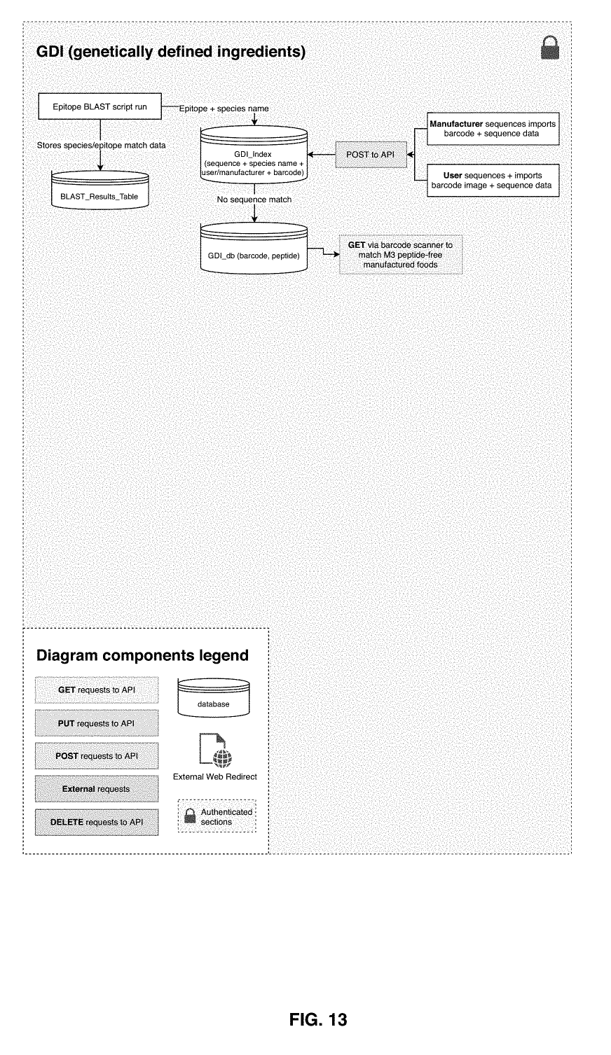

Embodiments of the foregoing and their advantages may be understood by referring to FIGS. 1-15, wherein like reference numerals refer to like elements.

In general, the following disclosure relates to a web-based or mobile application that sends and receives data related to genome sequencing, ancestry, and peptide presence in relation to foods and food groups. The application will source data either input from the user or inferred by ancestral lineage and determine if oral tolerance is developed due to antigenic peptides naturally occurring in food.

In use, the user will select from a series of tabs that each corresponding to a unique method of analysis including; universal, ancestry, prevention, react, and oncology. Each tab varies in the data required by the system and the data transmitted to and from the system. Further, each tab indicates a specific HLA allele assignment that corresponds to the depth of the search. In an embodiment, the user selects the universal tab, wherein all HLA alleles in a given database are searched. An epitope affinity table is generated based off of ic50 values corresponding to inferred alleles.

In an embodiment, the system clonal hematopoiesis of indeterminate potential (CHIP) as indications of cancer or viral disease.

In use, and in reference to FIG. 1, the user logs in by existing credentials, or creates new credentials according to predetermined system parameters. Credentials may be created from existing social media accounts, or genome database accounts. Data may be transmitted to and from each of the external networks to build profile information, or begin to infer a genotype related to the user. In an embodiment, the user will link their profile to one or more food, diet, supplement, or likewise network.

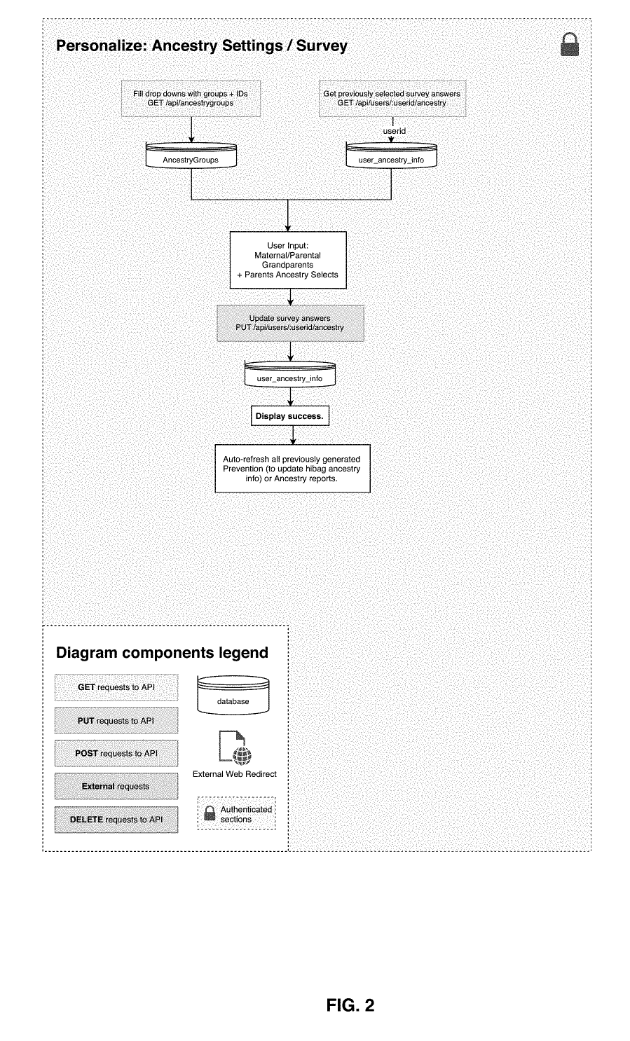

In reference to FIG. 2, a flowchart of a method of use is shown in regards to the generation of an ancestry survey from the system. In use, the user fills a series of drop down menus, or selects from a group of previously selected survey answers. As a result, ancestry groups or previously sourced user ancestry information is selected by the system. In an embodiment, the user inputs further maternal/parental ancestry information. The user places a request to the API to update survey answers. The system auto-refreshes all previously generated HLA imputation using attribute BAGging (hibag) ancestry information, or ancestry reports.

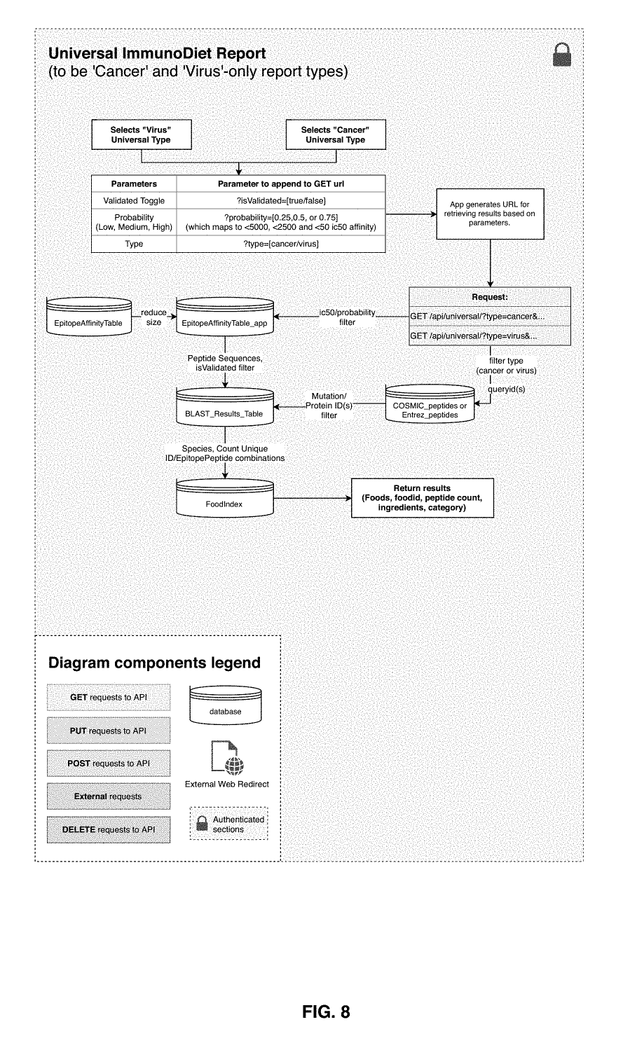

In an embodiment and in reference to FIGS. 8-13, peptides that are predicted to be useful in the recognition of a pathogen or cancerous cell may differ based on the HLA allele assignment of the user. Analysis begins with a computed totality of encoded peptides in either a virus or collection of cancer mutated proteins. These peptides are assigned ic50 values for HLA alleles. Based on the threshold of the ic50 values that corresponds to predicted affinity for the HLA alleles a subset of peptides is selected for the end user. This set of peptides composes the most likely epitopes useful for recognition of the cancer or virus infected tissue by T lymphocytes. A set of peptides may be considered that have been identified as immunogenic by alternative experimental testing in the present of T cells or ectopic T cell Receptor expressing cells, such as elispot, tetramer staining or tumor lysis assay or other similar assay system. Sources of these peptides are queried in the public database of the National Library of Medicine (NLM). In this embodiment, the BLAST system is used to locate likely sources of identical peptides in other organisms, genes, and proteins in which the peptide is encoded or has been physically identifies. The ‘BLAST system’ specifies the ‘lookup table’ part of the process. The system finds the short peptide sequences that should be avoided, and then we search in a database to see if it is contained in any food organism proteins. BLAST is a search algorithm that is built to access the continuously expanding national database of DNA and protein sequences. Each peptide can be found in 0,1, or 2+ non-human organisms. The sources of the peptides are filtered so that only potential edible sources of exposure are evaluated for the end user. For example, the presence of a peptide in a poisonous mushroom would not be included in the results. The species count from these results is used to inform the user of the highest risk foods. This count includes results of hits from peptides that are encoded in multiple genes. So 5 hits in Bos taurus can be from 5 different peptides that are encoded in 5 different cow genes. The count reflects an overall relative risk of exposure to peptides identified by the method above.

In an embodiment, the user selects an ancestry analysis wherein the top two most frequent HLA alleles in each of the users ancestry groups are assigned. The system accesses the users pre-downloaded, or external ancestry information that generates a set of alleles that meet a predetermined probability threshold that can then consult an epitope affinity table. In an embodiment, the user selects a prevention analysis, wherein HLA alleles are imputed from Chr6 rsID' s using the HiBag algorithm or other equivalent HLA imputation algorithm. The HiBag (HLA imputation using attribute BAGging) algorithm utilizes HLA and single nucleotide polymorphism (SNP) haplotype probabilities estimated from bootstrapped samples and SNP subsets. In an embodiment, the user selects the react analysis, wherein HLA alleles are assigned based on direct ngs sequencing of a sample. In an embodiment, the user selects the oncology analysis, wherein the HLA alleles are assigned based on direct ngs sequencing of a normal sample.

In an embodiment, seed and breed selection based on individual or population level epitope analysis is used to direct suggestions by the system. In an embodiment, data is used to compose animal and plant genomes that contain fewer cross-reactive peptides as identified by the system.

In an embodiment, the system is in communication with sequenced commercial food sources. For example, a fully inbred cattle heard, or individually sequenced animals could have a bar code on the packaging of the final food product. The bar code could be scanned by the user, allowing the user to evaluate a specific cow, or cattle heard for the specific user.

In an embodiment, the data can be used to identify DNA residues to be replaced in genetically modified organisms (GMO's) in order to avoid immunogenic peptides that may be present in foods as a result of genetic engineering.

In an embodiment, the system has calendared reminders of high-risk foods that relate to; specific seasons wherein the food is available, and specific locations wherein the food is available, as well as a combination thereof.

In an embodiment, the user is able to select one or more diseases or conditions of interest. For example, the user is aware that she has an increased risk of breast cancer. The system is able to identify epitopes with a high affinity for antigens commonly present in certain genetically modified chicken. The system alerts the user to this risk. Doing so reduces the chance for oral tolerance to develop, stemming from continued presentation of the antigen (if the user were to continue eating the genetically modified chicken). The system may also identify epitopes commonly present with her ancestry and identify other foods to avoid. For the sake of example, let's consider that the system identified oranges and oregano having antigenic proteins that have a high affinity to the users specific or imputed HLA alleles. The system then alerts the user to this, and provides diet manipulation options from external sources.

In an embodiment, the user selects a tab that allows the user to personalize their account. The user is directed to a heritage, or ancestry, survey. The user identifies ancestry groups located in a drop-down menu. In an embodiment, the user inputs ancestry information for maternal, paternal, grandparents, among other parameters. The system is able to access US census ancestry codes indexed with nation and international bone marrow transplant databases and anthropological studies.

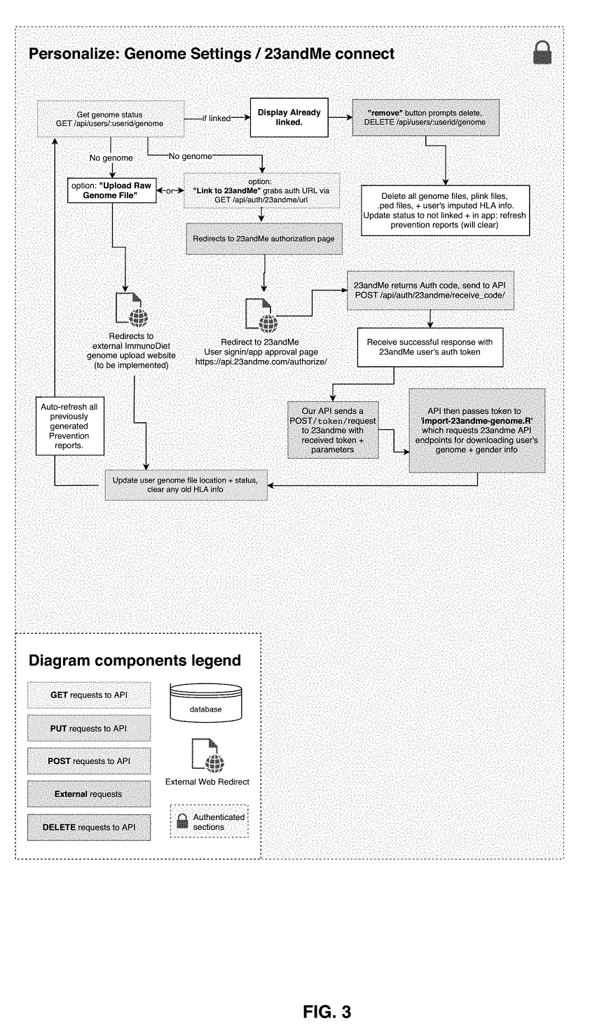

In an embodiment, the system is able to import and export data to and from an external network servers storing information related to the user. For example, the system can access 23andMe™ data. In specific reference to FIG. 3, the user can engage in up to three methods of the system. First, if a genome is already linked, the user has the option to view further details, or to input a delete request to the API. Second, if no genome is present, the user requests the API to link to 23andMe™ or other genome reference known in the art by utilizing an external request. The user is then redirected to the 23andMe™ network. Third, if no genome is present, the user can select to upload a raw genome file, wherein the user is redirected to external genome upload locations. In an embodiment, an authentication token is required to successfully link data within the API. The API executes the import of data once the authentication parameters are met. HLA alleles are based on SNP imputation algorithms known in the art.

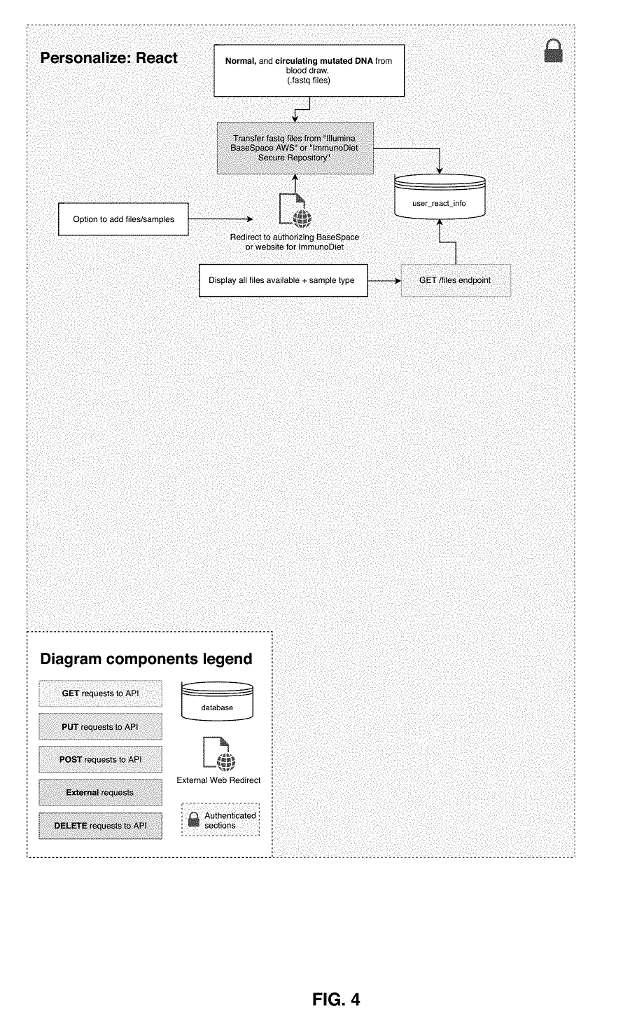

In an embodiment the user selects the react tab. In reference to FIG. 4, Results are displayed based on testing of circulating mutated DNA. For example, the system can access, by external request, a raw .fastq formatted next generation sequencing data from the Illumnia Basespace platform derived from sequencing of free floating or exome encased DNA or RNA. The system can identify sequences encoded known cancer mutation defining sequences and their quantities. The system can access prepared results from such analyses that provided a quantity of circulation mutated DNA from the user or users sequencing provider. New data is connected by the user and linked to their previous results, displaying changes in the amount of circulating mutated DNA. These changes are reflected in changes to their food avoidance report. Prioritized elimination recommendations are based on the mutated sequences that are increasing over time. Food elimination recommendations based on previously detected mutated DNA that are no longer present are removed from the elimination recommendation list if they are no longer indicated by other mutations that are still present.

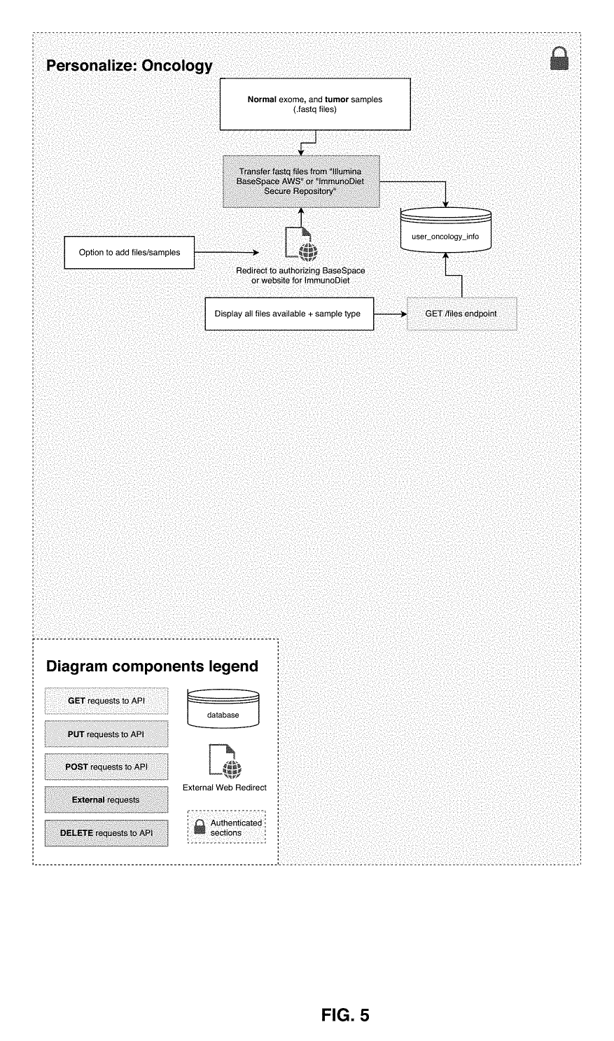

In reference to FIG. 5, the user inputs normal exome and tumor samples information. An external request is placed, wherein data is transmitted to an external network such as Illumina™ BaseSpace AWS™

The above description uses public domain algorithms in order to:

1. Determine known or predicted HLA alleles of an individual by imputation of HLA alleles from sparse genomic data.

2. Compute data from peptide: HLA affinity algorithms In an embodiment, nine different algorithms are used to generate ic50 scores. The lowest assigned score of the algorithms is used.

3. Determine digested peptides from food proteins according to germline DNA sequencing, RNA sequencing, or proteomic methods.

4. Determine acceptable or hazardous foods as well as related foods.

5. Consult a database of peptides associated with or predicted to be associated with immune responses.

In order to determine the likeliness of an antigenic peptide to be present in food, the system can use multiple methods to make a useful determination. In an embodiment, the system consults a species reference genome, single or multiple subspecies level measurements, and cultivar measurements.

Once the user has downloaded their ancestry report, genome, or other useful data, the system will generate a list of recommendations of foods and foodstuffs that may invoke oral tolerance. In an embodiment, the recommendations are merged with a meal planning system. In this embodiment, meal preparation plans are generated such that they avoid foods which have been identified as having a high likelihood of containing a peptide with a high epitope HLA affinity specific to the user.

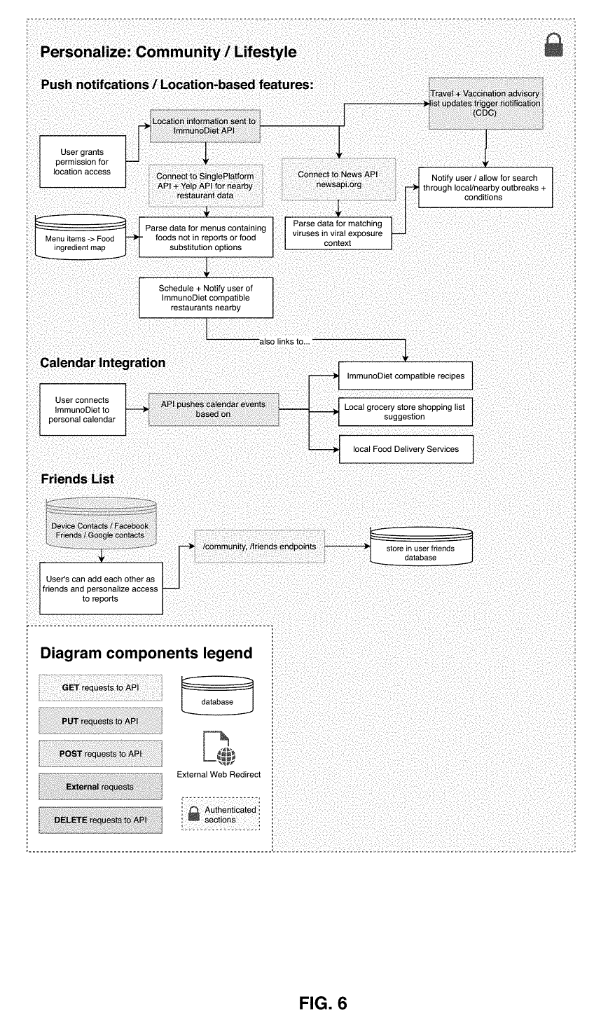

In reference to FIG. 6, the user selects a community and lifestyle tab, wherein the user can grant access to his/her location. A request is sent to the API to display location information. The user can then request from travel and vaccination advisory lists from one or more sources. The user is then notified of any alerts in their area and can also search through a list of nearby risks identified by the source (such as the CDC). In an embodiment, a request to the application programming interface (API) is sent to connect with an external network such as a news API. Data is then parsed from the news API for matching viruses in viral exposure context. Once more, notifications are sent to the user if significant parsed data is recognized.

In further reference to FIG. 6, menu items and food ingredient maps are stored in the database. Location data is requested, and if granted by the user, can be connected to single platform API, or external API's such as Yelp for restaurant data. Data is parsed from either the internal database, or external databases and API's such as Yelp. The API displays foods identified as containing user specific high affinity peptides and suggests substitutions, avoidance strategies, among other suggestions. In an embodiment, a schedule is implemented by the system notifying the user of compatible restaurants, seasonal foods, and regionally seasonal foods. This information can be linked to compatible recipes, grocery store shopping lists, local food delivery services, among others known in the art. In an embodiment, the user connects internal or external databases to a personal calendar. A request by the API to the user's calendar allows for reminders to be generated, logged, and sent to the user.

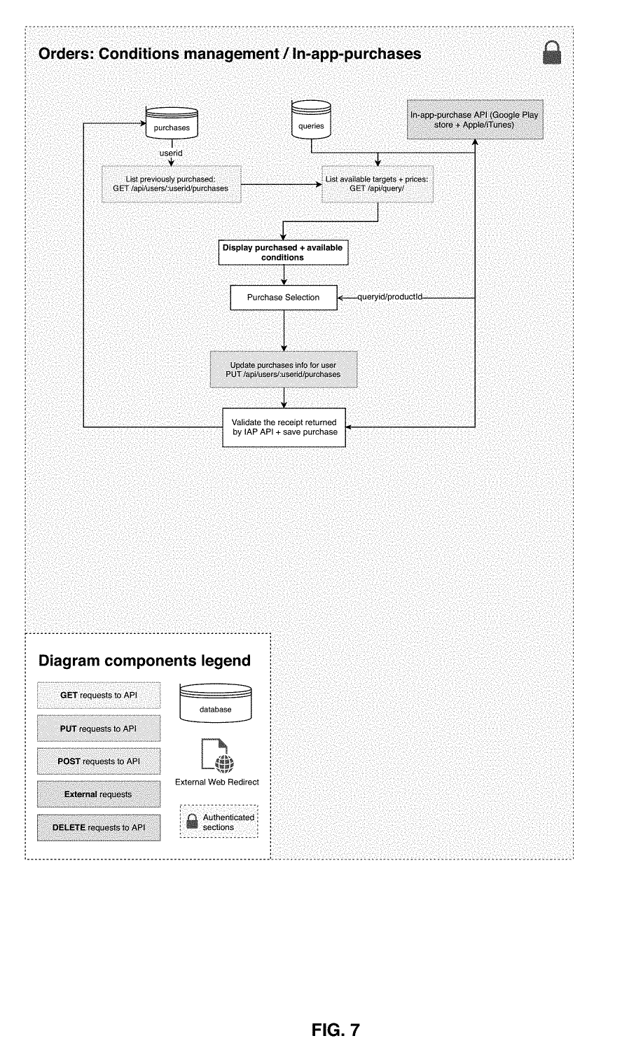

In reference to FIG. 7, the user is permitted to conduct in-app purchases as known in the art.

In an embodiment, the user can integrate a menu from one or more restaurants in order to automatically determine what menu items are less likely to contain the users specific high affinity peptide. Alternate embodiments comprise the system analyzing recipe websites, food blogs, photos of a recipe book, images of food, and other methods of food ingredient transmittal known in the art in order to identify the safest possible options in each form of food related media.

In an embodiment, the system can suggest food substitutions that are unlikely contain the users specific high affinity peptides. For example, if a specific GMO chicken is identified as hazardous, the system may highlight menu options, recipe options, or otherwise in which tofu is substituted for chicken.

In an embodiment, the system analyzes seed cultivars, GMO lines, among other food specifics in order to identify specific cultivars, lineages, regions, or species that should be avoided by the specific user.

In an embodiment, the system analyzes cancer treatment methods and suggests useful, hazardous, or ineffective cancer, immunotherapy, or likewise treatments based on the dietary history and practice of the patient.

In an embodiment, the system can be applied in the presence or absence of information about the individual T-Cell mediated immune response (TCR) repertoire recognition.

In an embodiment, the system can use multiple HLA peptide affinity prediction algorithms in order to select for peptides with reduced affinity.

In an embodiment, after the system has identified food of concern, the system tabulates the results in low, medium, and high-risk tabs allowing the user to filter their results. The user can select any tab and scroll through the corresponding foods. The user can select any food on the list to gain information related to the food specific to the user. In a embodiment, selecting a food opens a menu, showing the percentage of peptides matched by the plurality of algorithms used. The menu will display a percentage of peptides, as well as suggest foods to avoid that may contain the ingredient related to the peptide. In an example, the algorithms identify “2% of peptides matched in this report are also found in cacao. Avoid food with ingredients such as cocoa paste, cocoa powered, fudge swirl”. Ingredient identification may be linked to regulated terms provided and supervised by the US FDA or other regulatory body overseeing the labeling of food stuffs.

In an embodiment, users can select from the various food categories presented by the system such as; dairy, meat, vegetables, spices, and others.

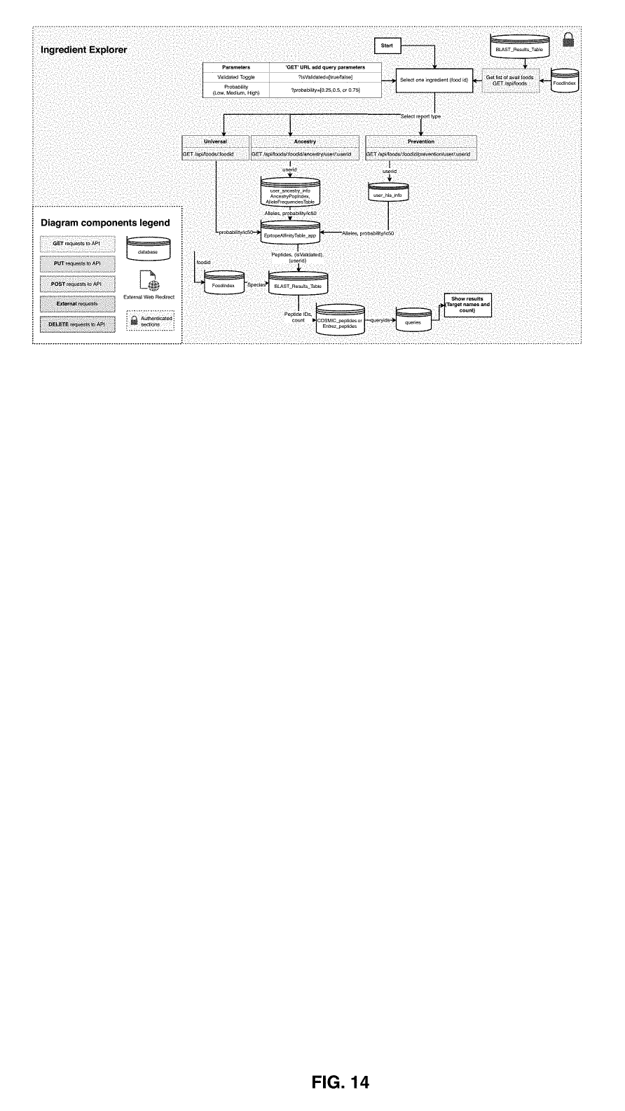

In reference to FIG. 14, the user selects at least one ingredient of food or selects from a list of available foods generated from a BLAST results table, or external food index. The user then selects a report type, including prevention, ancestry, or universal. Data from the selected food type or ingredient is input to one or more algorithms to determine results.

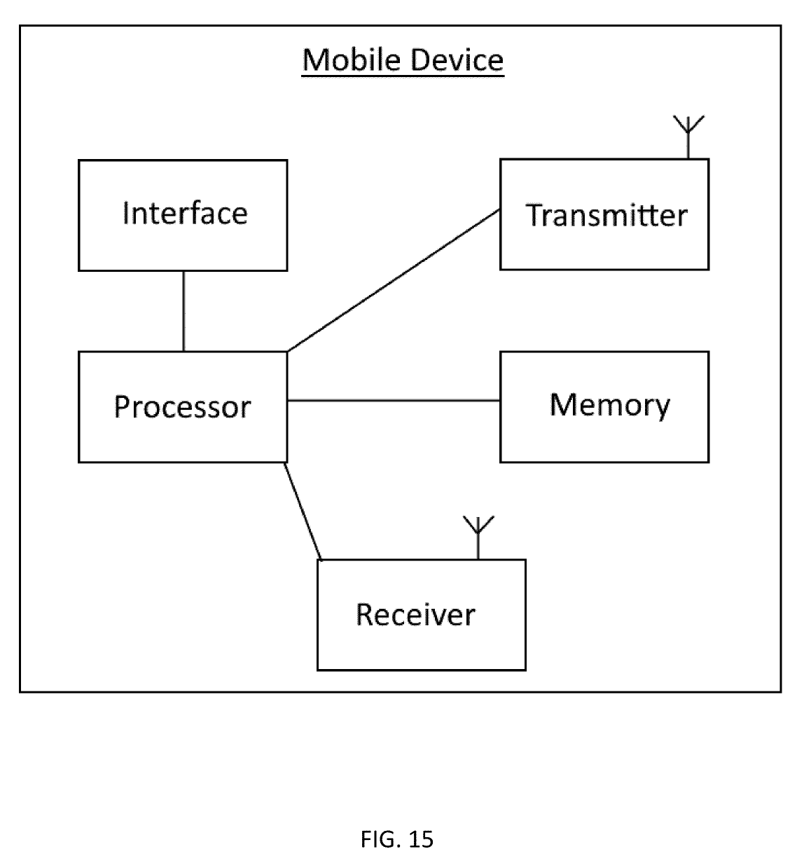

In some embodiments, the foregoing may be accomplished using a mobile device, such as a smart phone or tablet as shown in FIG. 15. The mobile device includes a user interface, such as an interactive screen or touchpad or keyboard for reading information from or entering information into the mobile device, one or more processors, for processing information and controlling the mobile device, a memory, connected to the one or more processors, for storing information. The functionality described above is contemplated as be accomplished on the mobile device or in connection with the mobile device accessing information which is remotely stored and processed.

The system of the embodiments presented or portions of the system thereof may be in the form of a “processing machine,” such as a general-purpose computer, for example. As used herein, the term “processing machine” is to be understood to include at least one processor that uses at least one memory. The at least one memory stores a set of instructions. The instructions may be either permanently or temporarily stored in the memory or memories of the processing machine. The processor executes the instructions that are stored in the memory or memories in order to process data. The set of instructions may include various instructions that perform a particular task or tasks, such as those tasks described above. Such a set of instructions for performing a particular task may be characterized as a program, software program, or simply software.

As noted above, the processing machine executes the instructions that are stored in the memory or memories to process data. This processing of data may be in response to commands by a user or users of the processing machine, in response to previous processing, in response to a request by another processing machine and/or any other input, for example.

As noted above, the processing machine used to implement some embodiments may be a general-purpose computer. However, the processing machine described above may also utilize any of a wide variety of other technologies including a special purpose computer, a computer system including, for example, a microcomputer, mini-computer or mainframe, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a programmable logic device (“PLD”) such as a Field-Programmable Gate Array (“FPGA”), Programmable Logic Array (“PLA”), or Programmable Array Logic (“PAL”), or any other device or arrangement of devices that is capable of implementing the steps of the processes described

The processing machine used to implement the foregoing may utilize a suitable operating system. Thus, embodiments of the foregoing may include a processing machine running the iOS operating system, the OS X operating system, the Android operating system, the Microsoft Windows™ 10 operating system, the Microsoft Window™ 8 operating system, Microsoft Windows™ 7 operating system, the Microsoft Windows™ Vista™ operating system, the Microsoft Windows™ XP™ operating system, the Microsoft Windows™ NT™ operating system, the Windows™ 2000 operating system, the Unix operating system, the Linux operating system, the Xenix operating system, the IBM AIX™ operating system, the Hewlett-Packard UX™ operating system, the Novell Netware™ operating system, the Sun Microsystems Solaris™ operating system, the OS/2™ operating system, the BeOS™ operating system, the various Apple iphone and MacOS operating systems, the Apache operating system, an OpenStep™ operating system or another operating system or platform.

Further, various technologies may be used to provide communication between the various processors and/or memories, as well as to allow the processors and/or the memories of the foregoing to communicate with any other entity, i.e., so as to obtain further instructions or to access and use remote memory stores, for example. Such technologies used to provide such communication might include a network, the Internet, Intranet, Extranet, LAN, an Ethernet, wireless communication via cell tower or satellite, or any client server system that provides communication, for example. Such communications technologies may use any suitable protocol such as TCP/IP, UDP, or OSI, for example.

As described above, a set of instructions may be used in the processing of the foregoing. The set of instructions may be in the form of a program or software. The software may be in the form of system software or application software, for example. The software might also be in the form of a collection of separate programs, a program module within a larger program, or a portion of a program module, for example. The software used might also include modular programming in the form of object-oriented programming The software tells the processing machine what to do with the data being processed.

Further, it is appreciated that the instructions or set of instructions used in the implementation and operation of the foregoing may be in a suitable form such that the processing machine may read the instructions. For example, the instructions that form a program may be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processing machine, i.e., to a particular type of computer, for example. The computer understands the machine language.

Any suitable programming language may be used in accordance with the various embodiments of the foregoing. Illustratively, the programming language used may include assembly language, Ada, APL, Basic, C, C++, COBOL, dBase, Forth, Fortran, Java, Modula-2, Pascal, Prolog, REXX, Visual Basic, and/or JavaScript, for example. Further, it is not necessary that a single type of instruction or single programming language be utilized in conjunction with the operation of the system and method of the foregoing. Rather, any number of different programming languages may be utilized as is necessary and/or desirable.

Also, the instructions and/or data used in the practice of the embodiments may utilize any compression or encryption technique or algorithm, as may be desired. An encryption module might be used to encrypt data. Further, files or other data may be decrypted using a suitable decryption module, for example.

As described above, some embodiments may illustratively be embodied in the form of a processing machine, including a computer or computer system, for example, that includes at least one memory. It is to be appreciated that the set of instructions, i.e., the software for example, that enables the computer operating system to perform the operations described above may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in the embodiments may take on any of a variety of physical forms or transmissions, for example. Illustratively, the medium may be in the form of paper, paper transparencies, a compact disk, a DVD, an integrated circuit, a hard disk, a floppy disk, an optical disk, a magnetic tape, a RAM, a ROM, a PROM, an EPROM, a wire, a cable, a fiber, a communications channel, a satellite transmission, a memory card, a SIM card, or other remote transmission, as well as any other medium or source of data that may be read by processors.

Further, the memory or memories used in the processing machine that implement the foregoing may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

The foregoing has been described herein using specific embodiments for the purposes of illustration only. It will be readily apparent to one of ordinary skill in the art, however, that the principles of the described herein may be embodied in other ways. Therefore, the foregoing should not be regarded as being limited in scope to the specific embodiments disclosed herein, but instead as being fully commensurate in scope with the following claims.

I claim:

1. A computer-readable, non-transitory programmable product, for developing a diet, or suggesting food options and alternatives, comprising code executable by one or more processors, to cause the one or more processors to do the following:
determine, according to a method of analysis, known or predicted HLA alleles of a subject by imputation of HLA alleles from the genomic data;
compute data from peptide: HLA affinity for the subject;
determine digested peptides from food proteins according to methods consisting of germline DNA sequencing, RNA sequencing, proteomic methods and a combination thereof;
determine acceptable or hazardous foods based on the HLA alleles;
receiving information about the intended consumption of a food by the subject; and
provide an alert in connection with the identification of hazardous food identified for intended consumption by the subject.

2. The computer-readable, non-transitory programmable product as recited in claim 1, wherein the genomic data includes DNA data for the subject.

3. The computer-readable, non-transitory programmable product as recited in claim 1, wherein the genomic data includes RNA data for the subject.

4. The computer-readable, non-transitory programmable product as recited in claim 1, wherein known or predicted HLA alleles of a subject are further determined by imputation of HLA alleles from heritage or ancestry information.

5. The computer-readable, non-transitory programmable product as recited in claim 1, where the method of analysis is based on factors affecting the subject consisting of factors affecting other subjects universally, factors based on ancestry, factors based on preventive medical care criteria, factors based on food allergic reaction criteria, factors based on oncological criteria and combinations thereof.

6. The computer-readable, non-transitory programmable product as recited in claim 1, further comprising code for determining peptides, for the recognition of a pathogen or mutated cells, associated with or predicted to be associated with immune responses.

7. The computer-readable, non-transitory programmable product as recited in claim 1, further comprising code to generate calendared reminders identifying high-risk foods according to HLA alleles information for the subject.

8. A mobile device being operable to develop a diet, or suggest food options and alternatives, comprising
a processor;
a receiver and a transmitter connected to the processor, the receiver being configured to receive HLA alleles data for subject based on the genomic data;
a memory connected to the processor; the memory storing genomic data for a subject;
and a user interface for entering food for consumption by the user, the mobile device further being operable to indicate alerts or warnings based on identifying high-risk foods according to the HLA alleles information for the subject.

9. The mobile device being operable to develop a diet, or suggest food options and alternatives, as recited in claim 8, wherein the memory genomic data includes DNA data for the subject.

10. The mobile device being operable to develop a diet, or suggest food options and alternatives, as recited in claim 8, wherein the memory genomic data includes RNA data for the subject.

11. The mobile device being operable to develop a diet, or suggest food options and alternatives, as recited in claim 8, wherein known or predicted HLA alleles of a subject are further determined by a method of imputation of HLA alleles from heritage or ancestry information.

12. The mobile device being operable to develop a diet, or suggest food options and alternatives, as recited in claim 8, wherein the memory stores peptide information, for the recognition of a pathogen or mutated cells, associated with or predicted to be associated with immune responses, the mobile device being further operable to indicate alerts or warnings based identifying high-risk foods according to peptide information determined to be harmful for a subject based on the genomic data.
